(19) United States
(12) Patent Application Publication
(10) Pub. No.: US 2014/0180172 A1
(43) Pub. Date: Jun. 26, 2014

(54) STABILOMETER AND POSTURAL STABILITY EVALUATING METHOD

(71) Applicant: TANITA CORPORATION, Tokyo (JP)

(72) Inventor: Tomoka UCHIYAMA, Tokyo (JP)

(73) Assignee: TANITA CORPORATION, Tokyo (JP)

(21) Appl. No.: 13/830,179

(22) Filed: Mar. 14, 2013

(30) Foreign Application Priority Data
Dec. 26, 2012 (JP) 2012-283393

(51) Int. Cl. A61B 5/11

(52) U.S. Cl. CPC A61B 5/1116
USPC 600/595

(57) ABSTRACT

A stabilometer including: a platform; a load detecting unit; a time counting unit; an arithmetic section; and a biometric evaluating unit configured to evaluate postural stability of a test subject, wherein the arithmetic section includes a determining unit that determines when the test subject has placed one foot on the platform completely, and a determining unit that determines when the test subject with the one foot placed on the platform gets the other foot off of the ground, and the biometric evaluating unit evaluates the postural stability of the test subject from when the test subject has placed one foot on the platform completely to when the test subject with the one foot placed on the platform gets the other foot off of the ground on the basis of at least one of center of gravity locus data, load fluctuation data per unit time, and time data.

### BACKGROUND OF THE INVENTION

1. Field of the Invention

The present invention relates to a stabilometer configured to be capable of evaluating postural stability by measuring how a measured person gets onto a measuring apparatus and a postural stability evaluating method using the stabilometer.

2. Description of the Related Art

Examples of a method of measuring postural stability of a test subject (user) and evaluating a state of disease of balance disorder in the related art include a balance disorder evaluating method disclosed in JP-A-7-250821. Disclosed as the balance disorder evaluating method is a method of evaluating the state of disease of balance disorder of the test subject on the basis of a value obtained by calculating a position of center of gravity of the test subject, converting the position of center of gravity into a position on a preset X-Y coordinate and obtaining a locus of the position of center of gravity with time, calculating a total locus length of the obtained locus, calculating a surface area of a locus figure inside an outermost circumferential line formed by the locus, and calculating a value of L/D where L is the total locus length and D is the surface area of the locus figure inside the outermost circumferential line. The stabilometer starts detection after the test subject has maintained a standstill posture on a detection panel (platform).

However, according to JP-A-7-250821, for example, if the test subject is an aged person, or has a disease, since he or she may take time to reach a standstill state on the detection panel, or may have difficulty to keep standstill, it may take a long time to start the measurement. In this manner, if it takes a long time to start the measurement, the measurement by itself imposes a burden to the test subject.

### SUMMARY

Accordingly, it is an object of the invention to provide a stabilometer capable of reducing a period required for measurement and a burden imposed on a test subject by measuring a load and the center of gravity corresponding to the time elapsed in a process from a state in which the test subject starts to get on a measuring apparatus until a time point when the test subject places his or her both feet on a detection panel and reaches stabilization.

In view of the problem described above, according to a first aspect of the invention, there is provided a stabilometer including: a platform on which a test subject gets on; a load detecting unit configured to detect a load applied to the platform at least at three points; a time counting unit configured to count time; an arithmetic section configured to calculate center of gravity locus data on the basis of the load detected by the load detecting unit and time data counted by the time counting unit; and a biometric evaluating unit configured to evaluate postural stability of the test subject, wherein the arithmetic section includes a determining unit configured to determine a time point when the test subject has placed one foot on the platform completely (Tb), and a determining unit configured to determine a time point when the test subject in a state in which the one foot is placed on the platform gets the other foot off of the ground (Tc), and the biometric evaluating unit evaluates the postural stability of the test subject during a period from the time point when the test subject has placed the one foot on the platform completely (Tb) to the time point when the test subject in a state in which the one foot is placed on the platform gets the other foot off of the ground (Tc) on the basis of at least one of the center of gravity locus data, fluctuation data of the load per unit time, and the time data.

Preferably, the arithmetic section includes a determining unit configured to determine a time point when the test subject has placed both feet on the platform completely (Td) and a determining unit configured to determine a time point when the test subject achieves a stable standstill standing position on the platform (Te), and the biometric evaluating unit evaluates the postural stability of the test subject during a period from the time point when the test subject has placed both feet on the platform completely (Td) to the time point when the test subject achieves a stable standstill standing position on the platform (Te) on the basis of at least one of the center of gravity locus data, fluctuation data of the load per unit time, and the time data.

Preferably, the biometric evaluating unit evaluates the postural stability relating to a dynamic balance of the test subject on the basis of at least one of the center of gravity locus data, fluctuation data of the load per unit time, and the time data obtained during a period from the time point when the test subject has placed the one foot on the platform completely (Tb) to the time point when the test subject in a state in which the one foot is placed on the platform gets the other foot off of the ground (Tc), and evaluates the postural stability relating to a static balance of the test subject on the basis of at least one of the center of gravity locus data, the fluctuation data of the load per unit time, and the time data obtained during a period from the time point when the test subject has placed both feet on the platform completely (Td) to the time point when the test subject achieves a stable standstill standing position on the platform (Te).

Preferably, the biometric evaluating unit performs evaluation synthetically on the basis of the respective results of evaluation of the postural stability during a period from the time point when the test subject has placed the one foot on the platform completely (Tb) to the time point when the test subject in a state in which the one foot is placed on the platform gets the other foot off of the ground (Tc), and a period from time point when the test subject has placed both feet on the platform completely (Td) to the time point when the test subject achieves a stable standstill standing position on the platform (Te).

According to a second aspect of the invention, there is provided a stabilometer including: a platform on which a test subject gets on; a load detecting unit configured to detect a load applied to the platform at least at three points; a time counting unit configured to count time; an arithmetic section configured to calculate center of gravity locus data on the basis of the load detected by the load detecting unit and time data counted by the time counting unit; and a biometric evaluating unit configured to evaluate postural stability of the test subject, wherein the arithmetic section includes a determining unit configured to determine a time point when the test subject starts to bring one foot into contact with the platform (Ta), a determining unit configured to determine a time point when the test subject has placed the one foot on the platform completely (Tb), and a determining unit configured to determine a time point when the test subject in a state in which the one foot is placed on the platform gets the other foot off of the ground (Tc), and the biometric evaluating unit evaluates the postural stability of the test subject during a period from the time point when the test subject starts to bring one foot into contact with the platform (Ta) to the time point when the test subject has placed the one foot on the platform completely (Tb), and a period from the time point when the test subject has placed the one foot on the platform completely (Tb) to the time point when the test subject in a state in which the one foot is placed on the platform gets the other foot off of the ground (Tc) on the basis of at least one of the center of gravity locus data, fluctuation data of the load per unit time, and the time data.

Preferably, the biometric evaluating unit performs evaluation synthetically on the basis of the respective results of evaluation of the postural stability during the period from the time point when the test subject starts to bring one foot into contact with the platform (Ta) to the time point when the test subject has placed the one foot on the platform completely (Tb), and the period from the time point when the test subject has placed the one foot on the platform completely (Tb) to the time point when the test subject in a state in which the one foot is placed on the platform gets the other foot off of the ground (Tc).

According to a third aspect of the invention, there is provided a stabilometer including: a platform on which a test subject gets on; a load detecting unit configured to detect a load applied to the platform at least at three points; a time counting unit configured to count time: an arithmetic section configured to calculate center of gravity locus data on the basis of the load detected by the load detecting unit and time data counted by the time counting unit; and a biometric evaluating unit configured to evaluate postural stability of the test subject, wherein the arithmetic section includes a determining unit configured to determine a time point when the test subject has placed one foot on the platform completely (Tb), a determining unit configured to determine a time point when the test subject in a state in which the one foot is placed on the platform gets the other foot off of the ground (Tc), and a determining unit configured to determine a time point when the test subject has placed both feet on the platform completely (Td), and the biometric evaluating unit evaluates the postural stability of the test subject during the period from the time point when the test subject has placed the one foot on the platform completely (Tb) to the time point when the test subject in a state in which the one foot is placed on the platform gets the other foot off of the ground (Tc), and the period from the time point when the test subject in a state in which the one foot is placed on the platform gets the other foot off of the ground (Tc) to the time point when the test subject has placed both feet on the platform completely (Td) on the basis of at least one of the center of gravity locus data, fluctuation data of the load per unit time, and the time data.

Preferably, the biometric evaluating unit performs evaluation synthetically on the basis of the respective results of evaluation of the postural stability during the period from the time point when the test subject has placed the one foot on the platform completely (Tb) to the time point when the test subject in a state in which the one foot is placed on the platform gets the other foot off of the ground (Tc) and during the period from the time point when the test subject in a state in which the one foot is placed on the platform gets the other foot off of the ground (Tc) to the time point when the test subject has placed both feet on the platform completely (Td).

According to a fourth aspect of the invention, there is provided a stabilometer including: a platform on which a test subject gets on; a load detecting unit configured to detect a load applied to the platform at least at three points; a time counting unit configured to count time: an arithmetic section configured to calculate center of gravity locus data on the basis of the load detected by the load detecting unit and time data counted by the time counting unit; and a biometric evaluating unit configured to evaluate postural stability of the test subject, wherein the arithmetic section includes a determining unit configured to determine a time point when the test subject starts to bring one foot into contact with the platform (Ta), a determining unit configured to determine a time point when the test subject has placed the one foot on the platform completely (Tb), a determining unit configured to determine a time point when the test subject in a state in which the one foot is placed on the platform gets the other foot off of the ground (Tc), a determining unit configured to determine a time point when the test subject has placed both feet on the platform completely (Td), and a determining unit configured to determine a time point when the test subject achieves a stable standstill standing position on the platform (Te), and the biometric evaluating unit evaluates the postural stability of the test subject during a period from the time point when the test subject starts to bring one foot into contact with the platform (Ta) to the time point when the test subject has placed the one foot on the platform completely (Tb), a period from the time point when the test subject has placed the one foot on the platform completely (Tb) to the time point when the test subject in a state in which the one foot is placed on the platform gets the other foot off of the ground (Tc), a period from the time point when the test subject in a state in which the one foot is placed on the platform gets the other foot off of the ground (Tc) to the time point when the test subject has placed both feet on the platform completely (Td), and a period from the time point when the test subject has placed both feet on the platform completely (Td) to the time point when the test subject achieves a stable standstill standing position on the platform (Te) on the basis of at least one of the center of gravity locus data, fluctuation data of the load per of the load unit time, and the time data.

Preferably, the biometric evaluating unit evaluates the postural stability relating to a dynamic balance of the test subject on the basis of at least one of the center of gravity locus data, fluctuation data of the load per unit time, and the time data obtained during a period from the time point when the test subject starts to bring one foot into contact with the platform (Ta) to the time point when the test subject has placed both feet on the platform completely (Td), and evaluates the postural stability relating to a static balance of the test subject on the basis of at least one of the center of gravity locus data, fluctuation data of the load per unit time, and the time data obtained during a period from the time point when the test subject has placed both feet on the platform completely (Td) to the time point when the test subject achieves a stable standstill standing position on the platform (Te).

Preferably, the biometric evaluating unit performs evaluation synthetically on the basis of the respective results of evaluation of the postural stability during a period from the time point when the test subject starts to bring one foot into contact with the platform (Ta) to the time point when the test subject has placed the one foot on the platform completely (Tb), a period from the time point when the test subject has placed the one foot on the platform completely (Tb) to the time point when the test subject in a state in which the one foot is placed on the platform gets the other foot off of the ground (Tc), a period from the time point when the test subject in a state in which the one foot is placed on the platform gets the other foot off of the ground (Tc) to the time point when the test subject has placed both feet on the platform completely (Td), and a period from the time point when the test subject has placed both feet on the platform completely (Td) to the time point when the test subject achieves a stable standstill standing position on the platform (Te).

Preferably, the stabilometer further includes a proximity detecting unit configured to detect whether or not the test subject enters a range at a predetermined distance with respect to the platform, and detection of a load by the load detecting unit and measurement of time by the time measuring unit are started when the proximity detecting unit detects the fact that the test subject enters the range at a predetermined distance with respect to the platform.

According to a fifth aspect of the invention, there is provided a postural stability evaluating method comprising:

a determination process b for determining a time point when a test subject has placed one foot on a platform of a stabilometer completely (Tb); a determination process c for determining a time point when the test subject in a state in which the one foot is placed on the platform gets the other foot off of the ground (Tc); and a biometric evaluating process for evaluating postural stability of the test subject on the basis of at least one of center of gravity locus data, fluctuation data of the load per unit time, and time data during a period from the time point when the test subject has placed the one foot on the platform of the stabilometer completely (Tb) to the time point when the test subject in a state in which the one foot is placed on the platform gets the other foot off of the ground (Tc).

According to the invention, since the test subject performs the evaluation of the postural stability on the basis of the motion getting on the platform of the stabilometer and the measurement for the evaluation of the postural stability may be completed already at a time point when the both feet are placed on the platform and stabilization is achieved, the measurement may be started immediately in comparison with the stabilometer of the related art which starts the measurement for the evaluation of the postural stability after having achieved a standstill posture on the platform. Accordingly, the time required for the measurement may be reduced, and hence the burden imposed on the test subject may be reduced.

### BRIEF DESCRIPTION OF THE PREFERRED EMBODIMENT

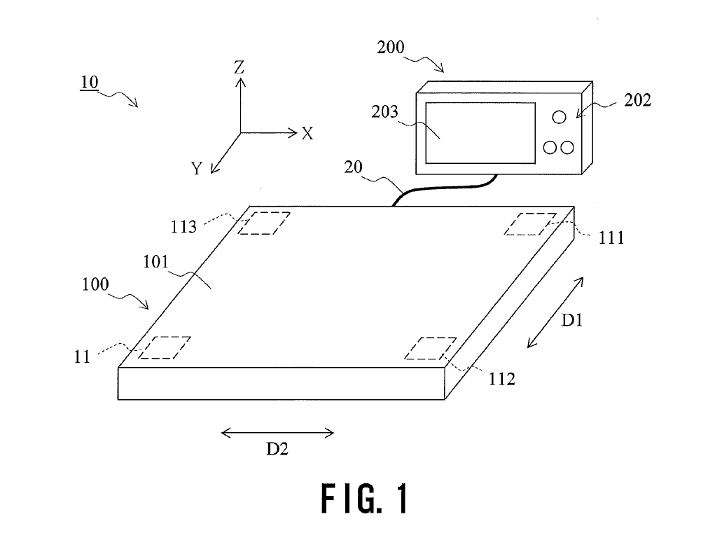

FIG. 1 is a perspective view illustrating an appearance configuration of a stabilometer according to an embodiment of the invention;

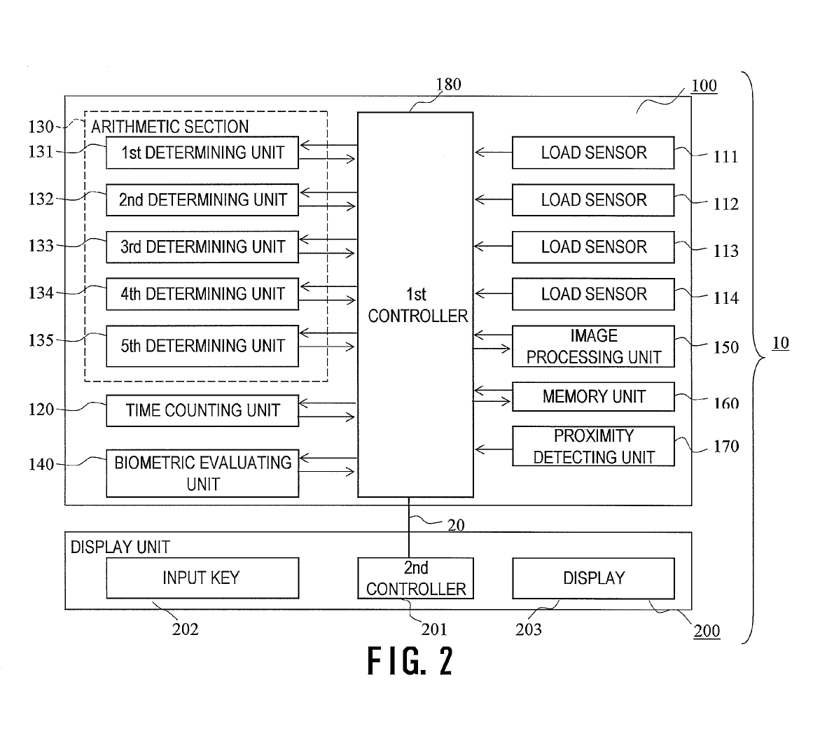

FIG. 2 is a block diagram illustrating a configuration of the stabilometer according to the embodiment of the invention;

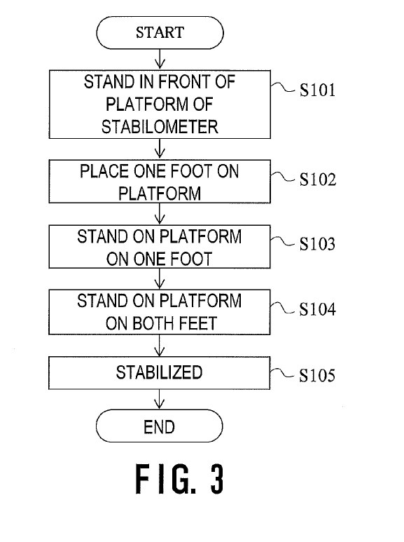

FIG. 3 is a flowchart illustrating a flow of motions of a test subject in a postural stability measurement process of the embodiment of the invention;

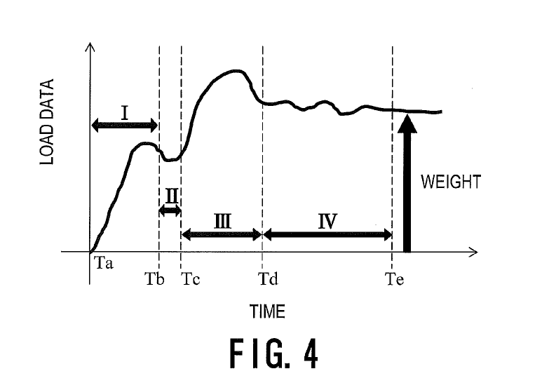

FIG. 4 is a graph illustrating an example of change of load data in the postural stability measurement process of the embodiment of the invention with respect to time;

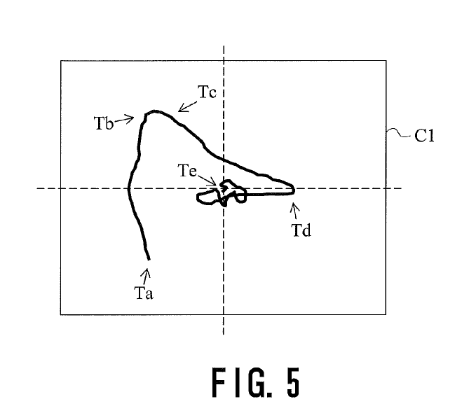

FIG. 5 is a drawing illustrating an example of the center of gravity locus data in the postural stability measurement process of the embodiment of the invention;

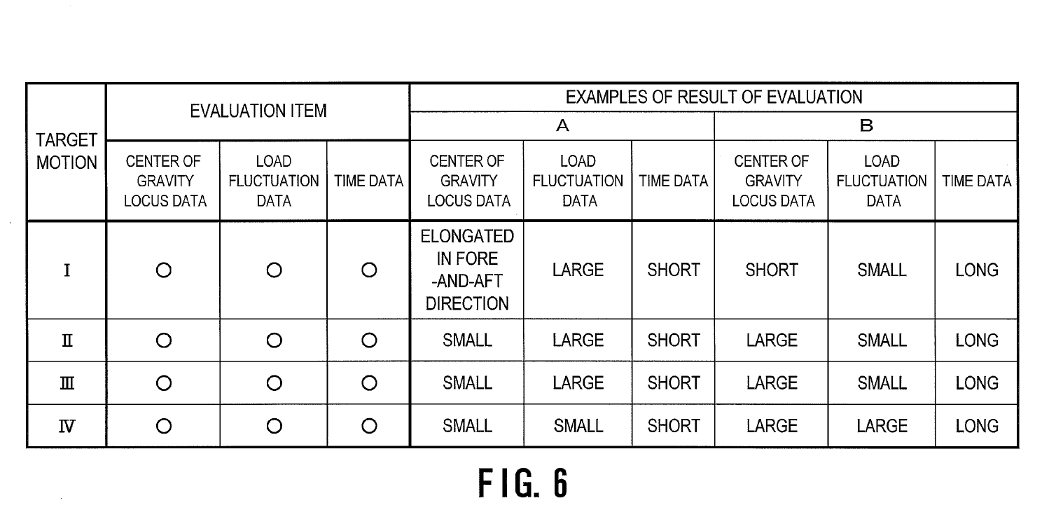

FIG. 6 is a table illustrating target motions, evaluation items, and examples of result of evaluation of the postural stability measurement process of the embodiment of the invention;

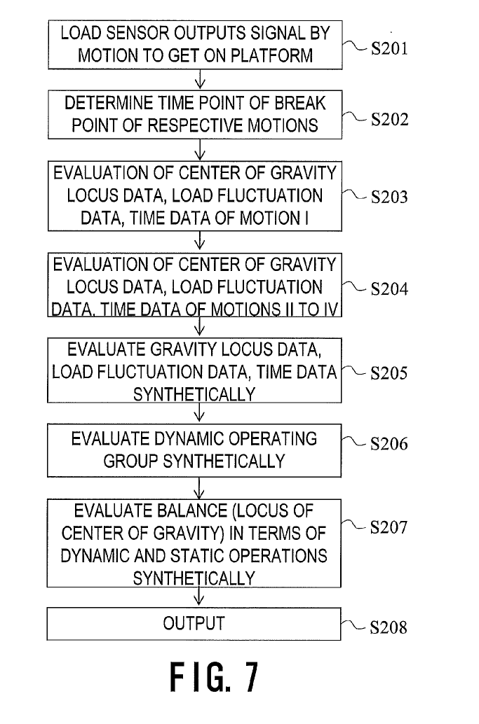

FIG. 7 is a flowchart illustrating a flow of the postural stability measurement process of the embodiment of the invention;

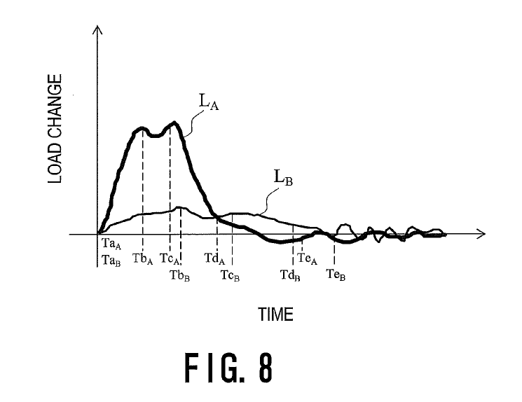

FIG. 8 is a graph illustrating an example of a load change per unit time with respect to time;

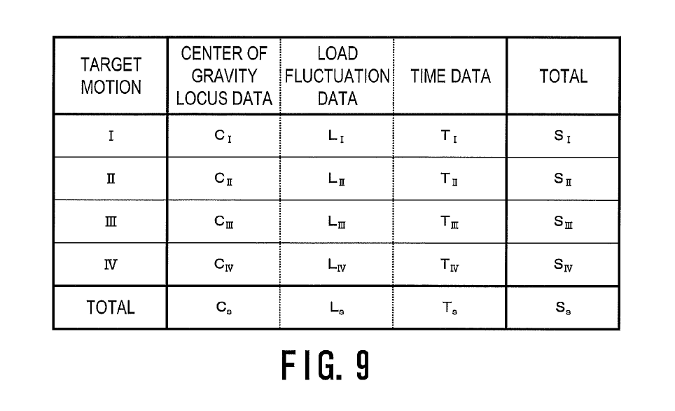

FIG. 9 is a table illustrating an example in which evaluation in the postural stability measurement process is expressed by scores;

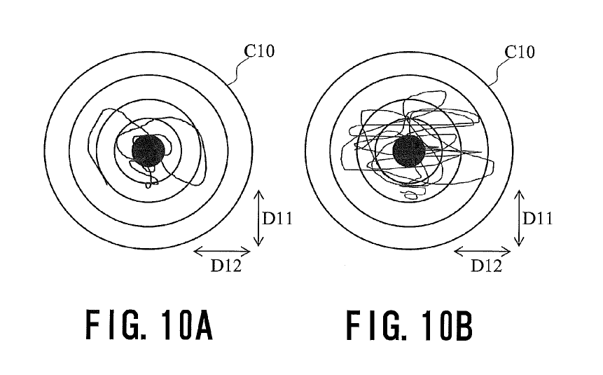

FIGS. 10A and 10B are drawings illustrating examples of results of measurement of the locus of center of gravity;

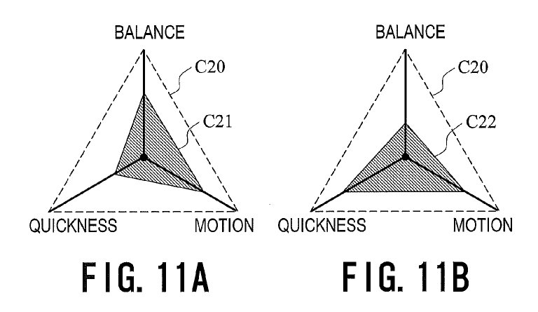

FIGS. 11A and 11B are drawings illustrating results of the evaluation in the postural stability measurement process from view points of balance, quickness, and movement; and

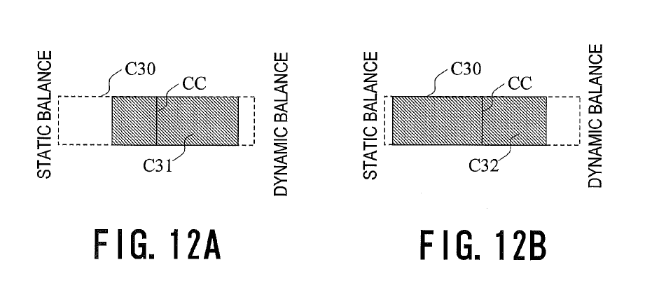

FIGS. 12A and 12B are drawings illustrating the results of evaluation in the postural stability measurement process from view points of static balance and dynamic balance.

### DESCRIPTION OF PREFERRED EMBODIMENT

Referring now to the drawings, a stabilometer according to embodiments of the invention will be described in detail.

FIG. 1 is a perspective view illustrating an appearance configuration of a stabilometer 10 according to an embodiment of the invention, and FIG. 2 is a block diagram illustrating a configuration of the stabilometer according to this embodiment.

As illustrated in FIG. 1 and FIG. 2, the stabilometer 10 includes a platform 100 and a display unit 200.

The platform 100 includes a horizontal measuring surface 101 on which a test subject gets on.

As a load detecting unit configured to detect a load applied to the platform 100 at least at three points, load sensors 111, 112, 113, and 114 are arranged at four corners of the platform 100 in this embodiment. Here, the load sensor 111 is arranged at an upper right corner when viewed from a direction vertical to the measuring surface 101, the load sensor 112 is arranged at a lower right corner, the load sensor 113 is arranged on the upper left corner, and the load sensor 114 is arranged at a lower left corner. The respective load sensors may be configured of a load cell used for measurement of body weight, but not specifically limited, and is configured to generate and output a detection signal according to a load applied vertically to a portion of the measuring surface 101 where a user by himself or herself is placed. The output signal is output to a first controller 180 and is used for postural stability measurement, described later. Although detailed illustration is omitted, the load cell may be configured to have a flexure element deformed according to the load input thereto and a skew gauge stuck to the flexure element and configured to output an electric signal (detection signal) of a value according to the deformation of the flexure element. The stabilometer 10 according to the embodiment has four load detecting units in the description. However, the number of the load detecting units is not specifically limited as long as the load is detected at three or more points. Postural stability evaluated by the stabilometer 10 of the invention includes postural stability on a horizontal surface 100a of the platform 100 (XY-axis in FIG. 1) and postural stability in the direction of gravitational force (Z-axis in FIG. 1).

As illustrated in FIG. 1, the display unit 200 includes various input keys 202 and a display 203. When the test subject or the like operates the input key 202, a command or data is entered into the display unit 200 according to the operation of the input key 202. Various items of information are displayed on the display 203. The displayed information includes, for example, data on measured load and elapsed time, an image drawing illustrating a locus of center of gravity, and a result of evaluation of the postural stability measurement. In this embodiment, a configuration provided with the display unit 200 will be described. However, the configuration is not specifically limited thereto, and information may be displayed on a display device as an external apparatus instead of the display unit 200.

As illustrated in FIG. 2, the first controller 180 is integrated in the platform 100. The first controller 180 is an arithmetic processing unit configured to execute various control processes, and performs overall-control the stabilometer 10.

As illustrated in FIG. 2, the load sensors 111, 112, 113, and 114, a time counting unit 120, an arithmetic section 130, a biometric evaluating unit 140, an image processing unit 150, a memory unit 160, and a proximity detecting unit 170 are connected to the first controller 180.

The time counting unit 120 is a circuit configured to count time. Counting of time is configured to be started according to the result of detection of the proximity detecting unit 170 under control of the first controller 180.

The memory unit 160 is a memory circuit, and is configured to store an input result input by the input key 202, data required for the postural stability measurement, and a result of measurement and a result of evaluation of the postural stability measurement.

The arithmetic section 130 includes a determining unit configured to determine a time point when the test subject performs a predetermined motion. In this embodiment, the arithmetic section 130 is an arithmetic circuit including a first determining unit 131, a second determining unit 132, a third determining unit 133, a fourth determining unit 134, and a fifth determining unit 135. The first determining unit 131, the second determining unit 132, the third determining unit 133, the fourth determining unit 134, and the fifth determining unit 135 are configured by individual arithmetic circuits or by a common arithmetic circuit.

The arithmetic section 130 calculates center of gravity locus data on the basis of load data detected by the respective load sensors 111, 112, 113, and 114 and time data counted by the time counting unit 120. The arithmetic section 130 calculates fluctuation data of the load per unit time on the basis of the load data and the time data.

The biometric evaluating unit 140 evaluates postural stability of the test subject and, more specifically, evaluates the postural stability of the test subject in all or some of a period from a time point (Ta) determined by the first determining unit 131 to a time point (Tb) determined by the second determining unit 132, a period from the time point (Tb) to a time point (Tc) determined by the third determining unit 133, a period from the time point (Tc) to a time point (Td) determined by the fourth determining unit 134, and a period from the time point (Td) to a time point (Te) determined by the fifth determining unit 135 on the basis of at least one of the center of gravity locus data, fluctuation data of the load per unit time, and the time data. In this embodiment, the stabilometer 10 configured to evaluate the postural stability of the test subject in all of the above-described periods will be described. However, the stabilometer 10 may be configured to evaluate the postural stability in any one of the above-described periods. In such a case, a configuration only needs a determining unit (any one of the first determining unit 131 to the fifth determining unit 135) for determining the period to be evaluated. The evaluation of the postural stability is an evaluation relating mainly to the physical strength (the state of the body) of the test subject, and includes, for example, physical capabilities of the body, having a muscle strength sufficient for stably supporting the body, having no disabling condition and having good health, and having no problem in nerve system.

The image processing unit 150 performs image processing for deploying the center of gravity locus data calculated by the arithmetic section 130 or the result of evaluation calculated by the biometric evaluating unit 140 as a predetermined virtual coordinate or an image, and displaying on the display 203.

The proximity detecting unit 170 detects the fact that the test subject enters a range at a predetermined distance from the platform 100. The proximity detecting unit 170 is not specifically limited, and an infrared ray sensor, for example, may be employed. The range at the predetermined distance to be detected by the proximity detecting unit 170 is preferably set to a distance suitable for determining the test subject's intent to be measured rationally, for example, a range from 10 to 15 cm.

The time counting unit 120 starts detection of load by the load detecting unit (the respective load sensors 111, 112, 113, and 114) and measurement of time by the time counting unit 120 when when the proximity detecting unit 170 detects the fact that the test subject enters the range described above.

It is also possible to provide a detecting unit configured to be capable of detecting whether or not power is distributed by contact of a foot (naked foot) of the test subject with two or more electrodes mounted on the platform 100 instead of the proximity detecting unit 170, and configured to start detection of load by the load detecting unit (the respective load sensors 111, 112, 113, and 114) and counting of time by the time counting unit 120.

As illustrated in FIG. 2, a second controller 201 is integrated in the display unit 200. The input keys 202 and the display 203 are connected to the second controller 201. The second controller 201 is connected to the first controller 180 in the platform 100 via a cable 20. Accordingly, transmission of signals between the second controller 201 and the first controller 180 is enabled.

When the test subject operates the input key 202, the height or other various data or command of the test subject are input to the second controller 201. The second controller 201 controls the display 203 according to the input data or command, or transmits the data or the command to the first controller 180. The first controller 180 executes various control processes according to the data or command received from the second controller 201.

Subsequently, measurement of the postural stability of the test subject (the change of the center of gravity with time) will be described.

FIG. 3 is a flowchart illustrating a flow of motion of the test subject in the postural way measurement process of this embodiment, FIG. 4 is a graph illustrating an example of change of load data in the postural way measurement process of this embodiment with respect to time, and FIG. 5 is a drawing of an example illustrating the center of gravity locus data in the postural way measurement process of this embodiment. FIG. 4 and FIG. 5 illustrate an example of a case where the test subject is a healthy young person.

When an evaluation of the center of gravity using the stabilometer 10 of this embodiment, the test subject performs a predetermined motion in a flow illustrated in FIG. 3. The test subject stands in front of the platform 100 of the stabilometer 10 (Step S101). At this time, the proximity detecting unit 170 detects that the test subject enters the range of a predetermined distance with respect to the stabilometer 10, and the postural way measurement process is started. When the process is started, the time counting unit 120 starts the time counting, and the display 203 displays a guide for the motions to be performed to the test subject, prompts the test subject to place one foot on the platform 100 one by one, and takes a standstill standing position on the platform 100.

Accordingly, the test subject places one foot (one of the feet) on the platform 100 (Step S102), stands on the platform 100 with both feet (Step S104) via a state of standing on the platform 100 on one foot with the remaining foot (the other foot) raised from the ground and kept in the air (Step S103), and takes a stable standstill standing position on the platform 100 (Step S105). The stabilometer 10 determines the motion of the test subject as described above at a time point when the test subject starts to bring one foot into contact with the platform 100 (Ta), a time point when the test subject has placed the one foot on the platform 100 completely (Tb), a time point when the test subject in a state in which the one foot is placed on the platform 100 gets the other foot off of the ground (Tc), a time point when the test subject has placed both feet on the platform 100 completely (Td), and a time point when the test subject achieves a stable standstill standing position on the platform 100 (Te) and performs the following evaluation.

Here, the foot to be placed on the platform 100 first may be any of left foot and right foot. How the test subject places the feet on the platform 100 is arbitrary and, for example, may place the foot so as to be grounded either from the heel side or from the toe side, or so that the entire part of the bottom of the foot is grounded simultaneously.

The first determining unit determines the time point when the test subject starts to bring the one foot into contact with the platform 100 (Ta) (a determination process a). Whether or not the one foot of the test subject starts to come into contact with the platform 100 is determined on the basis of a time point when the load sensors 111, 112, 113, and 114 start to detect the change of the load with respect to the measuring surface 101 along with the time data measured by the time counting unit 120.

The second determining unit determines the time point when the test subject has placed the one foot on the platform 100 completely (Tb) in Step S102 (a determination process b). In other words, the time point (Tb) corresponds to a time point when the fact that the test subject has finished to bring the entire part of the bottom of the one foot into contact with the platform 100 is detected. Whether placing of the one foot on the platform 100 is completed or only part of the bottom of the foot (for example, only the heel) is in contact with the platform 100 is determined on the basis of output signals from the load sensors 111, 112, 113, and 114 and the time data counted by the time counting unit 120.

The third determining unit determines the time point when the test subject in a state in which the one foot is placed on the platform 100 gets the other foot off of the ground (Tc) in Step S103 (a determination process c). In other words, the time point (Tc) corresponds to a time point when the test subject in the state in which the one foot is placed on the platform 100 stands on the corresponding one foot on the platform 100. Whether or not the other foot gets off of the ground is determined on the basis of the output signals from the load sensors 111, 112, 113, and 114 and the time data counted by the time counting unit 120.

The fourth determining unit determines the time point when the test subject has placed the both feet on the platform 100 completely (Td) in Step S104 (a determination process d). Whether or not the both feet are placed on the platform 100 is determined on the basis of the output signals from the load sensors 111, 112, 113, and 114 and the time data counted by the time counting unit 120.

The fifth determining unit determines the time point when the test subject achieves the stable standstill standing position on the platform 100 (Te) (a determination process e). The fifth determining unit determines the stable state of the test subject on the platform 100 on the basis of the center of gravity locus data, fluctuation data of the load per unit time, or the time data after the time point (Tc) whether the values of these data fall within the predetermined range.

As exemplified in FIG. 4, the load data changes with time according to the motions of the test subject. Here, the vertical axis in FIG. 4 represents load data relating to the entire platform 100 calculated from the respective load data detected respectively by the load sensors 111 to 114 through a known method, and a lateral axis represents time.

In FIG. 4, reference sign Ta denotes the time point when the test subject starts placing one foot on the platform 100. Reference sign Tb denotes the time point when the test subject has placed the one foot on the platform 100 completely, and reference sign Tc denotes the time point when the test subject in a state in which the one foot is placed on the platform 100 gets the other foot off of the ground. Reference sign Td denotes the time point when the test subject has placed the both feet on the platform 100 completely, and reference sign Te denotes the time point when the test subject achieves a stable standstill standing position on the platform 100. A load at the time point Te corresponds to the weight value of the test subject.

As exemplified in FIG. 5, a center of gravity locus is drawn according to the motions of the test subject. FIG. 5 illustrates the center of gravity locus calculated from the respective load data detected respectively by the load sensors 111 to 114 through the known method plotted on a coordinate C1 corresponding to the measuring surface 101 of the stabilometer 10. The upper side of the paper plane of FIG. 5 corresponds to the side where the load sensors 111 and 113 are arranged, and the lower side of the paper plane thereof corresponds to the side where the load sensors 112 and 114 are arranged, and an example in which the test subject gets on the measuring surface 101 from the left foot is illustrated. In other words, when to bring one foot into contact with the platform (Ta) the test subject starts to get on the platform 100 from the heel of the left foot (Ta), has placed the entire part of the left foot on the platform 100 completely (Tb), gets the right foot off of the ground and stands on one foot, that is, on the left foot (Tc), and has placed also the right foot on the platform 100 completely (Td), and takes a stable standstill standing position on the platform 100 (Te), the center of gravity locus as illustrated in FIG. 5 is obtained.

In the postural stability measurement, the following target motions I to IV illustrated in FIG. 6 are evaluated. FIG. 6 is a table illustrating target motions, evaluation items, and examples of results of evaluation of the postural stability measurement process of this embodiment.

Target motion I: from the time point when the test subject starts to bring one foot into contact with the platform 100 (Ta) to the time point when the test subject has placed the one foot on the platform 100 completely (Tb) (Step S101 to S102 in FIG. 3, see I in FIG. 4). The motion I detects whether or not the test subject is landed from the toe or from the heel, or, alternatively, landed with the entire part of the bottom of the foot focusing on a landing motion onto the platform 100 for legs as an object to be evaluated.

Target motion II: from the time point when the test subject has placed the one foot on the platform 100 completely (Tb) to the time point when the test subject in a state in which the one foot is placed on the platform 100 gets the other foot off of the ground (Tc) (Step S102 to S103 in FIG. 3, see II in FIG. 4). The target to be evaluated of the target motion II is a motion to step on the one foot already placed on the platform 100.

Target motion III: from the time point when the test subject in a state in which the one foot is placed on the platform 100 gets the other foot off of the ground (Tc) to the time point when the test subject has placed the both feet on the platform 100 completely (Td) (Step S103 to S104 in FIG. 3, see III in FIG. 4). The target to be evaluated of the motion III is a motion to draw the other foot got off of the ground from the state of standing on one foot, and the swinging of the body, the locus of center of gravity thereby, and application of the load (the magnitude and time) are detected.

Target motion IV: from the time point when the test subject has placed both feet on the platform 100 completely (Td) to the time point when the test subject achieves a standstill standing position on the platform 100 (Te) (Step S104 to S105 in FIG. 3, see IV in FIG. 4). The target to be evaluated of the motion IV is a motion from a state immediately after having started to stand on the both feet until the standstill standing position is achieved, and time required for maintaining the stable posture, if the knees or the low back is bent, time required for stretching knees or the low back if the knees or the low back is bent, and postural stability or acceleration in association therewith are detected.

How the test subject walks, or how fast the test subject walks may be estimated from the feet landing motion of the above-described motion I. For example, if the translation of the center of gravity is significant, it is estimated physically that the test subject is a young person.

In the above-described motion II, the speed of movement and the stability may be evaluated from the period during which the test subject stands on the one foot, and the sway occurring during that period.

In the above-described motion IV, time and swaying motion (the fore-and-aft direction or the left-and-right direction) until the standstill standing position is achieved is estimated to be equivalent to the normal stable standstill posture maintaining properties.

Furthermore, sway in the vertical direction (the direction of center of gravity) is considered to be quicker if the acceleration is larger. If the movement is slow, disorder of the low back, the knees, or the feet is contemplated. If there is a significant difference between left and right, disorder of the knees or the feet may be estimated. By specifying the direction of getting on the platform 100, how the feet are placed and how much the body swayed may be estimated from the fluctuation of the pressure distribution.

Therefore, by evaluating the above-described motions I to IV, the dynamic ability may be estimated from the view points of speed of the motions, stability, controlling ability, lateral balance, and the like. In addition, disorder of the body such as pain of joints (knees or low back) may be seen from the motion and the lateral balance. By interpreting data (normal postural stability) after having maintained the standstill posture, s measurement and evaluation of the dynamic and static equilibrium sense are achieved simultaneously.

Here, the evaluation of the postural stability of the test subject may be performed on the basis of all or some of the motions from among the motion I, the motion II, the motion III, and the motion IV. However, in the description of this embodiment, an example in which the postural stability is evaluated in terms of all of the motions I to IV will be described.

In FIG. 6, three items of the center of gravity locus data, fluctuation data of the load (fluctuation data of the load per unit time), and the time data are illustrated as items to be evaluated. However, the motions I to IV may be evaluated on the basis of at least one of these items. In this embodiment, an example in which the evaluation is performed in terms of all of the three items will be described.

By considering the center of gravity locus data (cm), the smoothness of the motions, the balance (if the deflections in the lateral direction or in the fore-and-aft direction is large or small), and whether or not the motion is correct may be evaluated. Here, the motions showing a locus of center of gravity different from the person having a physical strength in terms of the deflection of the deviation in center of gravity is interpreted as a motion showing a decline of power for supporting the body or a motion being protective toward some abnormal portion. When evaluating the center of gravity locus data, height data of the test subject is preferably taken into consideration.

By taking fluctuation data of the load (fluctuation data of load per unit time) into consideration, the magnitude of the motion, whether or not the test subject is capable of moving quickly or at a normal speed, whether or not the rhythm of the motion is abnormal, and so forth may be evaluated. For example, when the load fluctuation per unit time is small, it may be determined to be a slow motion. As regards the rhythm of the motion, if an abrupt load fluctuation is detected during the slow motion, it may be determined that the test subject cannot control his or her motion well.

The time data (ms) helps to evaluate time required for a predetermined motion.

The evaluation of the motion is performed for each evaluation item such as the center of gravity locus data, fluctuation data of the load, and the time data.

As regards the center of gravity locus data, having the long locus of center of gravity in the fore-and-aft direction is highly appreciated in the motion I, and having the small deviations in the locus of center of gravity in the lateral and for-and-aft directions and having the short locus to the next motion are highly appreciated in the motions II to IV. The term “highly appreciated” means to evaluate the test subject to have a higher physical strength (the same applies, hereinafter).

As regards fluctuation data of the load, having the large fluctuation data of the load per unit time and/or per weight is highly appreciated in the motions I to III, and having the small load fluctuation is highly appreciated in the motion IV.

As regards the time data, finishing the respective motions in a short time is highly appreciated.

The results of evaluation of the respective evaluation items; the center of gravity locus data, fluctuation data of the load, and the time data are preferably scored (see FIG. 9) or, in addition, are preferably evaluated as a total score for each of the motions I to IV. The scoring of each of the evaluation items is performed in one-by-one correspondence to the evaluation entries (for example, the balance, the disorder of the feet or the low back, legerity) of the evaluation items.

In FIG. 6, “A” corresponds to a case of a healthy young person (a test subject having a high physical strength) and “B” corresponds to a case of an aged person (a test subject having low physical strength). Hereinafter, an example of the result of evaluation illustrated in FIG. 6 will be described.

In the motion I, the test subject “A” was evaluated to have a long locus of center of gravity in the fore-and-aft direction, while the test subject “B” was evaluated to have a short locus of center of gravity. Here, the fore-and-aft direction means the direction along a direction D1 in FIG. 1, and corresponds to the fore-and-aft direction of the test subject on the platform 100. The lateral direction means the direction along a direction D2 in FIG. 1.

In the motion I, the test subject “A” was evaluated to have a large load fluctuation and take a short time, while the test subject “B” was evaluated to have a small load function and take a long time.

In the motions II and III, the test subject “A” was evaluated to have a small locus of center of gravity, a large load fluctuation, and take a short time elapsed, while the test subject “B” was evaluated to have a large locus of center of gravity, a small load fluctuation, and take a long time elapsed. From the results as described above, the test subject “A” is interpreted to be brisk in motion, and present less wobbling as regards the motions I to III.

In the motion IV, the test subject “A” was evaluated to have a small locus of center of gravity, a small load fluctuation, and take a short time elapsed, while the test subject “B” was evaluated to have a large locus of center of gravity, a large load fluctuation, and take a long time elapsed. Accordingly, the test subject “B” is understood to be unstable and present larger wobbling on the platform 100, and take time before reaching the stable state as regards the motion IV.

Subsequently, the process to be performed by the stabilometer 10 according to the target motions I to IV of the test subject and the evaluation obtained by this process will be described with reference to FIG. 7. FIG. 7 is a flowchart illustrating a flow of the postural stability measurement process of this embodiment.

When the test subject stands in front of the stabilometer 10 and performs the target motions I to IV in sequence, loads are applied to the load sensors 111, 112, 113, and 114 according to the motion of the test subject getting on the platform 100, and corresponding detection signals from the respective load sensors are output to the arithmetic section 130 via the first controller 180 (Step S201).

The arithmetic section 130 (the first determining unit 131 to the fifth determining unit 135) that receives the detection signals from the respective load sensors coordinates the detection signals and the time signals counted by the time counting unit 120 into one-to-one correspondence, and determines the break time points among the motions I to IV of the test subject (the time point (Ta), the time point (Tb), the time point (Tc), the time point (Td), and the time point (Te)) from the detection signals and the changes thereof (Step S202).

The biometric evaluating unit 140 evaluates the target motion I in terms of the center of gravity locus data, fluctuation data of the load, and the time data by a predetermined method (Step S203). The evaluation is performed by using expressions and tables stored in advance in the memory unit 160. The result of evaluation is preferably scored because the comparison among the test subjects is enabled.

Subsequently, the biometric evaluating unit 140 evaluates the target motions II to IV in terms of the center of gravity locus data, fluctuation data of the load, and the time data by a predetermined method (Step S204). The evaluation is performed by using expressions and tables stored in advance in the memory unit 160. The result of evaluation is preferably scored because the comparison among the test subjects is enabled.

The biometric evaluating unit 140 then evaluates synthetically to the test subject on the basis of the result of evaluation of the respective evaluation items in the steps S203 and S204 (Step S205). The evaluation synthetically includes, but not limited to, calculation of the total score by adding the scores obtained in the Step S203 and S204, for example.

Subsequently, the biometric evaluating unit 140 performs evaluation synthetically on the dynamic motion group (the target motions I to III) (Step S206), and performs evaluation of the balance synthetically (the center of gravity locus) with the dynamic motion and the static motion (Step S207). Finally, the biometric evaluating unit 140 outputs the result of evaluation obtained in the steps described above to the display 203 or the like (see FIG. 10A to FIG. 12B). In this embodiment, at least the steps S205 and S206 correspond to biometric evaluation steps.

The above-described evaluation will be described with more detailed examples with reference to FIG. 8 to FIG. 12.

FIG. 8 is a graph illustrating an example of a load change per unit time (vertical axis) with respect to time (lateral axis). In FIG. 8, a line L_{A} represents a load change in a case where a test subject A is a healthy young person (a test subject having a physical strength), and a line L_{B} represents a load change in a case of a debilitated test subject B (a test subject having a low physical strength). In the same manner as in FIG. 4, the time point when the test subject starts to bring one foot into contact with the platform 100 (Ta) is set to zero on the lateral axis. Reference signs Ta (Ta_{A}, Ta_{B}), Tb (Tb_{A}, Tb_{B}), Tc (Tc_{A}, Tc_{B}), Td(Td_{A}, Td_{B}), Te(Te_{A}, Te_{B}) are the same as those in FIG. 4, and represents the time point (Ta) to the time point (Te) determined in the above-described step S202, respectively. The vertical axis represents the load function data calculated by the above-described steps S203 and S204, which represents the load change per unit time, and the load change is reduced with the stabilization of the posture of the test subject.

When a period between Ta_{A} and Tb_{A} and a portion between Ta_{B} and Tb_{B} are compared, it is understood that the load fluctuation is large and the time required for the motion is short in the case of the healthy test subject A (L_{A}). Other periods as well, the difference between the both is obviously observed.

FIG. 9 is a table illustrating an example in which evaluation in the postural stability measurement process is expressed by scores. Specifically, FIG. 9 illustrates an example in which the evaluation result obtained in the above-described steps S203 to S205 is scored. In the same manner as FIG. 6, the center of gravity locus data, fluctuation data of the load, and the time data are evaluated in terms of the target motions I to IV, respectively, and is represented by scores C_{I} to C_{IV}, L_{I} to L_{Iv}, and T₁ to T_{IV}. Alternatively, evaluation may be achieved by calculation of the total score per evaluation item (C_{S}, L_{S}, T_{S}), calculation of the total score per target motion I to IV (S_{I}, S_{II}, S_{III}, S_{IV}), or calculation of the total score (S_{S}) for all of the evaluation items and all of the target motions. Here, the scores of the motions I to III correspond to evaluations of dynamic motion and the score of the motion IV corresponds to an evaluation of static motion. The score of the center of gravity locus data corresponds to an evaluation of balance, the score of fluctuation data of the load corresponds to an evaluation of motion, and the score of the time data corresponds to an evaluation of quickness.

FIGS. 10A and 10B are drawings illustrating examples of results of evaluation of the locus of center of gravity. In the examples illustrated in FIGS. 10A and 10B, the locus of the center of gravity is drawn in a frame C10 corresponding to the measuring surface 101 of the platform 100, and a direction D11 corresponds to the fore-and-aft direction D1 and a direction D12 corresponds to the left-and-right direction D12. FIG. 10A illustrates a case where the test subject is a healthy young person (the test subject having a high physical strength) and FIG. 10B illustrates a case where the test subject is an aged person (the test subject having a low physical strength).

From FIG. 10A, it is understood that the test subject has a high muscle strength, has placed his or her foot on the measuring surface 101 from the heel, and is stable in posture with little deviation. In contrast, from FIG. 10B, it is understood that the test subject is in a decline in muscle strength of the feet which supports the body, and has placed his or her foot on the measuring surface 101 so that the entire surface of the bottom of the foot comes into contact therewith at the same time, had a large deviation, and needed a long time to achieve the posture stabilization.

FIGS. 11A and 11B are drawings visualizing the result of the evaluation in the postural stability measurement process from view points of balance, quickness, and movement. In an example illustrated in FIGS. 11A and 11B, triangles C21 (FIG. 11A) and C22 (FIG. 11B) formed by connecting scores as the actual result of evaluation in a frame C20 formed by connecting maximum values of balance, quickness, and motion are displayed. The display of these triangles is performed by the image processing unit 150 on the basis of the score data. FIG. 11A illustrates an example of being superior in the balance and the magnitude of the motion but low in the degree of quickness, and FIG. 11B illustrates an example of being superior in quickness and the magnitude of the motion, but low in score of balance. When being visualized using such display, the physical strength of the test subject may be evaluated on the basis of the shape and the size of the triangle. The evaluation is not limited to visualization on the basis of the three points of view; balance, quickness, and motion, and may be expressed from one or more point of view.

FIGS. 12A and 12B are drawings visualizing the result of the evaluation in the postural stability measurement process from view points of static balance and the dynamic balance. The drawings illustrated in FIGS. 12A and 12B are creased by the image processing unit 150 on the basis of the scores of the locus of center of gravity relating to the balance from among scores illustrated in FIG. 9. The terms “dynamic balance” illustrated in FIGS. 12A and 12B each correspond to the total of the loci of center of gravity of the motions I to III, and the “static balance” corresponds to the score of the locus of center of gravity of the motion IV. The drawings in FIGS. 12A and 12B illustrate a rectangle C31 (FIG. 12A) and a rectangle C32 (FIG. 12B) which represent the balance of the scores in a frame C30 that covers the maximum values of the static balance and the dynamic balance. The positions of the rectangles C31, C32 are formed by calculating the ratio of the scores corresponding to the static balance and the dynamic balance by a predetermined calculating expression and are arranged with reference to a center position CC of the frame C30 on the basis of the calculated ratio. From these drawings, the balance between the static balance and the dynamic balance is visualized to enable the evaluation of the physical strength of the test subject. In FIG. 12A, the test subject may be evaluated to be good in dynamic balance, but has uncertainty in maintenance of the static posture. In FIG. 12B, the test subject may be evaluated to have uncertainty in motion to some extent, but be capable of maintaining the static posture.

In the configuration as described above, the following advantages are achieved according to the above-described embodiment.

(1) The evaluation of the physical strength (the physical capabilities) evaluated with a plurality of tests in the related art may be inspected by aiming at a series of motions performed once in a short time.

(2) The dynamically balancing capability and the statically balancing capability which have been required to measure separately in the related art may be evaluated simultaneously in a series of motions.

(3) The muscle strength and the balancing capability may be evaluated by measuring how the test subject gets on the platform to measure the dynamic and static physical capabilities simultaneously.

(4) The evaluation is achieved simply by a motion of getting on the platform, the time required for starting the measurement is reduced in comparison with the stabilometer of the related art, in which the measurement is started after the center of gravity of the test subject on the platform is stabilized, and hence a burden imparted on the test subject may be reduced.

(5) The motion of getting on the platform includes a variety of motions, and hence a larger number of items of information are obtained in comparison with the measuring method performed only by standing on the base as in the related art. In other words, information on the movement, getting up, speed and balance of the posture maintenance may be obtained.

A modification will be described below.

By using the load cells as the load sensors as described above, functions of a weighting machine, a BIA body composition meter, a body fat meter, a biometric information measuring apparatus may be integrated therein.

When assuming that the body composition meter and the weighting machine are integrated and that the test subject gets on the platform 100 with naked feed, counting of the elapsed time may be started by the load sensors 111 to 114 provided on the measuring surface 101 of the platform 100 or a contact sensor or a foot switch provided on a side surface of the platform 100 instead of the infrared ray sensor.

The load sensor may be embedded in a floor or in a footstep instead of on the platform.

By providing the platform with a certain height, the differences among individuals may be figured out further obviously. The height may be adjustable according to the height of the test subject.

In order to accommodate a case where the test subject uses a stick on a daily basis, by configuring the stabilometer to be capable of evaluating the state using the stick, the physical capabilities achieved by using the stick may also be evaluated.

The evaluation may be configured to determine the level in several steps (for example, “attention required”, “problematic”, “no problem.” instead of the scoring described above.

The determination may be performed synthetically from the balance (the locus of center of gravity) and the required time (including the load fluctuation).

The dynamic balance and the static balance may be evaluated separately.

The numerical values (locus length, load fluctuation, time) calculated for each of the evaluation items may be displayed in the form of the numerical values as results.

Although the invention has been described on the basis of the above-described embodiment, the invention is not limited to the above-described embodiment, and may be improved or modified within the scope of the object of the improvement and the spirit of the invention.

### INDUSTRIAL APPLICABILITY

As described thus far, the stabilometer and the postural stability evaluating method according to the invention allow the postural stability measurement to be performed with fewer burdens imposed on the test subject, whereby the physical strength of the test subject is effectively evaluated correctly and objectively.

What is claimed is:

1. A stabilometer comprising:
a platform on which a test subject gets on;
a load detecting unit configured to detect a load applied to the platform at least at three points;
a time counting unit configured to count time;
an arithmetic section configured to calculate center of gravity locus data on the basis of the load detected by the load detecting unit and time data counted by the time counting unit; and
a biometric evaluating unit configured to evaluate postural stability of the test subject, wherein
the arithmetic section includes a determining unit configured to determine a time point (Tb) when the test subject has placed one foot on the platform completely, and a determining unit configured to determine a time point (Tc) when the test subject in a state in which the one foot is placed on the platform gets the other foot off of the ground, and
the biometric evaluating unit evaluates the postural stability of the test subject during a period from the time point (Tb) when the test subject has placed the one foot on the platform completely to the time point (Tc) when the test subject in a state in which the one foot is placed on the platform gets the other foot off of the ground on the basis of at least one of the center of gravity locus data, fluctuation data of the load per unit time, and the time data.

2. The stabilometer according to claim 1, wherein
the arithmetic section includes a determining unit configured to determine a time point when the test subject has placed both feet on the platform completely and a determining unit configured to determine a time point when the test subject achieves a stable standstill standing position on the platform, and
the biometric evaluating unit evaluates the postural stability of the test subject during a period from the time point when the test subject has placed both feet on the platform completely to the time point when the test subject achieves a stable standstill standing position on the platform on the basis of at least one of the center of gravity locus data, fluctuation data of the load per unit time, and the time data.

3. The stabilometer according to claim 2, wherein the biometric evaluating unit
evaluates the postural stability relating to a dynamic balance of the test subject on the basis of at least one of the center of gravity locus data, fluctuation data of the load per unit time, and the time data obtained during a period from the time point (Tb) when the test subject has placed the one foot on the platform completely to the time point (Tc) when the test subject in a state in which the one foot is placed on the platform gets the other foot off of the ground, and
evaluates the postural stability relating to a static balance of the test subject on the basis of at least one of the center of gravity locus data, fluctuation data of the load per unit time, and the time data obtained during a period from the time point (Td) when the test subject has placed both feet on the platform completely to the time point (Te) when the test subject achieves a stable standstill standing position on the platform.

4. The stabilometer according to claim 2 or claim 3, wherein the biometric evaluating unit performs evaluation synthetically on the basis of the respective results of evaluation of the postural stability during a period from (Tb) the time point when the test subject has placed the one foot on the platform completely to the time point (Tc) when the test subject in a state in which the one foot is placed on the platform gets the other foot off of the ground, and a period from time point (Td) when the test subject has placed both feet on the platform completely to the time point (Te) when the test subject achieves a stable standstill standing position on the platform.

5. A stabilometer comprising:
a platform on which a test subject gets on;
a load detecting unit configured to detect a load applied to the platform at least at three points;
a time counting unit configured to count time;
an arithmetic section configured to calculate center of gravity locus data on the basis of the load detected by the load detecting unit and time data counted by the time counting unit; and
a biometric evaluating unit configured to evaluate postural stability of the test subject, wherein
the arithmetic section includes a determining unit configured to determine a time point (Ta) when the test subject starts to bring one foot into contact with the platform, a determining unit configured to determine a time point (Tb) when the test subject has placed the one foot on the platform completely, and a determining unit configured to determine a time point (Tc) when the test subject in a state in which the one foot is placed on the platform gets the other foot off of the ground, and
the biometric evaluating unit evaluates the postural stability of the test subject during a period from the time point (Ta) when the test subject starts to bring one foot into contact with the platform to the time point (Tb) when the test subject has placed the one foot on the platform completely, and a period from the time point (Tb) when the test subject has placed the one foot on the platform completely to the time point (Tc) when the test subject in a state in which the one foot is placed on the platform gets the other foot off of the ground on the basis of at least one of the center of gravity locus data, fluctuation data of the load per unit time, and the time data.

6. The stabilometer according to claim 5, wherein the biometric evaluating unit performs evaluation synthetically on the basis of the respective results of evaluation of the postural stability during the period from the time point (Ta) when the test subject starts to bring one foot into contact with the platform to the time point (Tb) when the test subject has placed the one foot on the platform completely, and the period from the time point (Tb) when the test subject has placed the one foot on the platform completely to the time point (Tc) when the test subject in a state in which the one foot is placed on the platform gets the other foot off of the ground.

7. A stabilometer comprising:
a platform on which a test subject gets on;
a load detecting unit configured to detect a load applied to the platform at least at three points;
a time counting unit configured to count time:
an arithmetic section configured to calculate center of gravity locus data on the basis of the load detected by the load detecting unit and time data counted by the time counting unit; and
a biometric evaluating unit configured to evaluate postural stability of the test subject, wherein
the arithmetic section includes a determining unit configured to determine a time point (Tb) when the test subject has placed one foot on the platform completely, a determining unit configured to determine a time point (Tc) when the test subject in a state in which the one foot is placed on the platform gets the other foot off of the ground, and a determining unit configured to determine a time point (Td) when the test subject has placed both feet on the platform completely, and
the biometric evaluating unit evaluates the postural stability of the test subject during the period from the time point (Tb) when the test subject has placed the one foot on the platform completely to the time point (Tc) when the test subject in a state in which the one foot is placed on the platform gets the other foot off of the ground, and the period from the time point (Tc) when the test subject in a state in which the one foot is placed on the platform gets the other foot off of the ground to the time point (Td) when the test subject has placed both feet on the platform completely on the basis of at least one of the center of gravity locus data, fluctuation data of the load per unit time, and the time data.

8. The stabilometer according to claim 7, wherein the biometric evaluating unit performs evaluation synthetically on the basis of the respective results of evaluation of the postural stability during the period from the time point (Tb) when the test subject has placed the one foot on the platform completely to the time point (Tc) when the test subject in a state in which the one foot is placed on the platform gets the other foot off of the ground and during the period from the time point (Tc) when the test subject in a state in which the one foot is placed on the platform gets the other foot off of the ground to the time point (Td) when the test subject has placed both feet on the platform completely.

9. A stabilometer comprising:
a platform on which a test subject gets on;
a load detecting unit configured to detect a load applied to the platform at least at three points;
a time counting unit configured to count time:
an arithmetic section configured to calculate center of gravity locus data on the basis of the load detected by the load detecting unit and time data counted by the time counting unit; and
a biometric evaluating unit configured to evaluate postural stability of the test subject, wherein
the arithmetic section includes a determining unit configured to determine a time point (Ta) when the test subject starts to bring one foot into contact with the platform, a determining unit configured to determine a time point (Tb) when the test subject has placed the one foot on the platform completely, a determining unit configured to determine a time point (Tc) when the test subject in a state in which the one foot is placed on the platform gets the other foot off of the ground, a determining unit configured to determine a time point (Td) when the test subject has placed both feet on the platform completely, and a determining unit configured to determine a time point (Te) when the test subject achieves a stable standstill standing position on the platform, and
the biometric evaluating unit evaluates the postural stability of the test subject during a period from the time point (Ta) when the test subject starts to bring one foot into contact with the platform to the time point (Tb) when the test subject has placed the one foot on the platform completely, a period from the time point (Tb) when the test subject has placed the one foot on the platform completely to the time point (Tc) when the test subject in a state in which the one foot is placed on the platform gets the other foot off of the ground, a period from the time point (Tc) when the test subject in a state in which the one foot is placed on the platform gets the other foot off of the ground to the time point (Td) when the test subject has placed both feet on the platform completely, and a period from the time point (Td) when the test subject has placed both feet on the platform completely to the time point (Te) when the test subject achieves a stable standstill standing position on the platform on the basis of at least one of the center of gravity locus data, fluctuation data of the load per unit time, and the time data.

10. The stabilometer according to claim 9, wherein the biometric evaluating unit evaluates the postural stability relating to a dynamic balance of the test subject on the basis of at least one of the center of gravity locus data, fluctuation data of the load per unit time, and the time data obtained during a period from the time point (Ta) when the test subject starts to bring one foot into contact with the platform to the time point (Td) when the test subject has placed both feet on the platform completely, and
evaluates the postural stability relating to a static balance of the test subject on the basis of at least one of the center of gravity locus data, fluctuation data of the load per unit time, and the time data obtained during a period from the time point (Td) when the test subject has placed both feet on the platform completely to the time point (Te) when the test subject achieves a stable standstill standing position on the platform.

11. The stabilometer according to claim 9 or claim 10, wherein the biometric evaluating unit performs evaluation synthetically on the basis of the respective results of evaluation of the postural stability during a period from the time point (Ta) when the test subject starts to bring one foot into contact with the platform to the time point (Tb) when the test subject has placed the one foot on the platform completely, a period from the time point (Tb) when the test subject has placed the one foot on the platform completely to the time point (Tc) when the test subject in a state in which the one foot is placed on the platform gets the other foot off of the ground, a period from the time point (Tc) when the test subject in a state in which the one foot is placed on the platform gets the other foot off of the ground to the time point (Td) when the test subject has placed both feet on the platform completely, and a period from the time point (Td) when the test subject has placed both feet on the platform completely to the time point (Te) when the test subject achieves a stable standstill standing position on the platform.

12. The stabilometer according to claim 1, further comprising a proximity detecting unit configured to detect whether or not the test subject enters a range at a predetermined distance with respect to the platform, wherein detection of a load by the load detecting unit and measurement of time by the time measuring unit are started when the proximity detecting unit detects the fact that the test subject enters the range at a predetermined distance with respect to the platform.

13. The stabilometer according to claim 5, further comprising a proximity detecting unit configured to detect whether or not the test subject enters a range at a predetermined distance with respect to the platform, wherein detection of a load by the load detecting unit and measurement of time by the time measuring unit are started when the proximity detecting unit detects the fact that the test subject enters the range at a predetermined distance with respect to the platform.

14. The stabilometer according to claim 7, further comprising a proximity detecting unit configured to detect whether or not the test subject enters a range at a predetermined distance with respect to the platform, wherein detection of a load by the load detecting unit and measurement of time by the time measuring unit are started when the proximity detecting unit detects the fact that the test subject enters the range at a predetermined distance with respect to the platform.

15. The stabilometer according to claim 9, further comprising a proximity detecting unit configured to detect whether or not the test subject enters a range at a predetermined distance with respect to the platform, wherein detection of a load by the load detecting unit and measurement of time by the time measuring unit are started when the proximity detecting unit detects the fact that the test subject enters the range at a predetermined distance with respect to the platform.

16. A postural stability evaluating method comprising:
a determination process b for determining a time point (Tb) when a test subject has placed one foot on a platform of a stabilometer completely;
a determination process c for determining a time point (Tc) when the test subject in a state in which the one foot is placed on the platform gets the other foot off of the ground; and
a biometric evaluating process for evaluating postural stability of the test subject on the basis of at least one of center of gravity locus data, load fluctuation data per unit time, and time data during a period from the time point (Tb) when the test subject has placed the one foot on the platform of the stabilometer completely to the time point (Tc) when the test subject in a state in which the one foot is placed on the platform gets the other foot off of the ground.
